(19) United States
(12) Patent Application Publication
(10) Pub. No.: US 2012/0237963 A1
(43) Pub. Date: Sep. 20, 2012

(54) APPARATUS AND METHOD FOR ASSESSING COMPOSTABILITY OR BIODEGRADABILITY

(75) Inventors: Cleston C. Lange, Farmington, MN (US); William K. Reagen, Stillwater, MN (US); Jeffrey W. Stock, Pine City, MN (US)

(73) Assignee: 3M Innovative Properties Company

(21) Appl. No.: 13/417,376

(22) Filed: Mar. 12, 2012

Related U.S. Application Data
(60) Provisional application No. 61/454,037, filed on Mar. 18, 2011.

(51) Int. Cl. C12Q 1/02; C12M 1/00

(52) U.S. Cl. 435/29; 435/289.1

(57) ABSTRACT

An apparatus and method for performing compostability tests that provides significantly more data and reveals trends much more quickly than previously described apparatus. In a first aspect, the invention provides an apparatus, comprising: a flowmeter for metering a flow of oxygen-containing gas to a humidifier; a bioreactor comprising a body, a gas inlet mounted on the body and connected to the humidifier, a dispersive element for distributing the flow of humidified gas throughout the body, and a gas outlet; a first moisture trap connected to the outlet a mass flow meter connected to the first moisture trap for accurately measuring the mass flow from the outlet; and a non-dispersive IR detector for measuring the carbon dioxide in the gas from the outlet. Convenient embodiments include a digitally controlled electronic manifold for sequentially directing gas flows from multiple reactors to the analyzer/detector.

### CROSS REFERENCE TO RELATED APPLICATION

This application claims the benefit of U.S. Provisional Patent Application No. 61/454,037, filed Mar. 18, 2011, the disclosure of which is incorporated by reference herein in its entirety.

### TECHNICAL FIELD

The present disclosure relates to the assessment of compostability or biodegradability of materials, and more specifically, to an apparatus and method for performing such assessments in an automated manner, more particularly using multiple specimens measured simultaneously.

### BACKGROUND

The use of compostable or biodegradable materials, for example packaging materials, product containers, and even consumer products themselves, can greatly ameliorate the problems of solid waste disposal in landfills. Tests have been developed to assess the ability of materials to biodegrade under conditions that simulate real world composting or landfill conditions. For example, ASTM D 5338-98 (2003), is designed to be used with all plastic materials that are not themselves inhibitory to the microorganisms present in aerobic composting piles. Exemplary apparatus and methods for evaluating the biodegradability or compostability of landfillable materials are known in the art, for example, U.S. Pat. Nos. 4,798,802 (Ryan); 5,320,807 (Brinton et al.); and 5,427,947 (Dalos).

### SUMMARY

Known apparatus and methods for performing composting or biodegradability testing, for example ASTM D 5338-98 (2003), are often slow to reveal a trend, time consuming, and difficult to use. The present disclosure provides, in some exemplary embodiments, an improved apparatus and method for performing compostability (i.e. biodegradability) tests such as ASTM standard D 5338-98 (2003). In certain exemplary embodiments, the apparatus and method are computer automated and provide significantly more data and reveal trends much more quickly than previously described apparatus.

Thus, in one aspect, the disclosure describes an apparatus, comprising:

a flow metering device for metering a flow of oxygen-containing gas to a humidifier,

a bioreactor comprising:

a body having an internal volume, a gas inlet connected with said bioreactor in flow communication between the humidifier and the internal volume to receive the flow of oxygen-containing gas in a humidified state from the humidifier, a dispersive element for distributing the flow of oxygen-containing gas in a humidified state throughout the internal volume, and a gas outlet in flow communication with the internal volume;

a first moisture trap in flow communication with the gas outlet;

a mass flow measuring device in flow communication with the first moisture trap; and

a non-dispersive infrared detector in flow communication with the mass flow measuring device, the non-dispersive infrared detector adapted to measure the concentration of carbon dioxide in a gas flowing from the first moisture trap.

In one exemplary embodiment, the apparatus further comprises:

at least three additional bioreactors, each additional bioreactor comprising:

a body having an internal volume, a gas inlet connected with said each additional bioreactor in flow communication between the humidifier and the internal volume to receive the flow of oxygen-containing gas in a humidified state from the humidifier,

a dispersive element for distributing the flow of oxygen-containing gas in a humidified state throughout the internal volume of the each additional bioreactor, and

a gas outlet in flow communication with the internal volume of the each additional bioreactor;

at least three additional first moisture traps, each additional first moisture trap in flow communication with the gas outlet of one of the each additional bioreactors; and

a multi-valve manifold in flow communication with each of the first moisture traps, the multi-valve manifold adapted to alternately direct gas from one of the first moisture traps in turn to the mass flow measuring device. In further exemplary embodiments of the foregoing embodiment, the apparatus further comprises a second moisture trap disposed between the multi-valve manifold and the non-dispersive infrared detector.

In some presently preferred embodiments, the apparatus will have at least three additional bioreactors and at least three additional first moisture traps connected to these bioreactors. Such an arrangement allows a sample, a blank, a positive control, and a negative control to be tested simultaneously. An electronically-controlled multi-valve manifold for alternately directing gas from the bioreactors in turn to the mass-flow detector and the non-dispersive IR detector may be present in these embodiments. In any of the preceding aspect and embodiments, the internal volume of each bioreactor is from about 2 to 4 liters, and further the flow metering device delivers from about 100 to 150 ml of air per minute at ambient temperature and pressure to each bioreactor.

In another aspect, the disclosure describes a method comprising:

(a) providing an apparatus comprising:
a flow metering device for metering a flow of oxygen-containing gas to a humidifier;
a bioreactor comprising a body having an internal volume, a gas inlet connected with said bioreactor in flow communication between the humidifier and the internal volume to receive the flow of oxygen-containing gas in a humidified state from the humidifier, a dispersive element for distributing the flow of oxygen-containing gas in a humidified state throughout the internal volume, and a gas outlet in flow communication with the internal volume;
a first moisture trap in flow communication with the gas outlet;
a mass flow measuring device in flow communication with the first moisture trap; and
a non-dispersive infrared detector in flow communication with the mass flow measuring device, the non-dispersive infrared detector adapted to measure the concentration of carbon dioxide in a gas flowing from the first moisture trap;

(b) adding to the internal volume of the bioreactor an at least partially compostable material, a liquid, and an inoculum comprising at least one bacterium;

(c) starting the flow of oxygen-containing gas to the bioreactor while maintaining the temperature of the bioreactor at a temperature sufficient to support asexual reproduction of the at least one bacterium, thereby producing a plurality of bacteria; and

(d) measure the concentration of carbon dioxide in the gas flowing from the first moisture trap over a time period sufficient to determine the rate at which the at least partially compostable material is digested by the plurality of bacteria.

In some presently preferred embodiments, the internal volume of each bioreactor is from about 2 to 4 liters, and further wherein the flow metering device delivers from about 100 to 150 ml of air per minute at ambient temperature and pressure to each bioreactor. In certain of these embodiments, the mass flow measuring device is an electronic flow meter disposed so as to measure the mass flow rate of the gas flowing from the first moisture trap to the non-dispersive infrared detector. In additional such embodiments, the method further comprises a computer receiving information from the mass flow measuring device and the non-dispersive infrared detector, wherein the computer uses the information to calculate and reports the rate at which the at least partially compostable material is digested by the plurality of bacteria.

Various aspects and advantages of exemplary embodiments of the disclosure have been summarized. The above Summary is not intended to describe each illustrated embodiment or every implementation of the present invention. The Drawings and the Detailed Description that follow more particularly exemplify certain preferred embodiments using the principles disclosed herein.

### BRIEF DESCRIPTION OF THE DRAWINGS

In the accompanying drawings:

FIG. 1 is a schematic of an exemplary apparatus according to the present disclosure;

FIG. 2 is an exploded perspective view of a bioreactor particularly adapted to the assessment of solid samples; and

FIG. 3 is an exploded perspective view of an alternate bioreactor particularly adapted to the assessment of liquid samples.

FIG. 4A is an exemplary plot of carbon dioxide concentration as a function of composting time determined using non-dispersive infrared monitoring of the dried gas stream exiting a bioreactor in which sucrose or glycerol are digested by bacteria obtained from a municipal sewage waste inoculum.

FIG. 4B is an exemplary plot of mass percent of the sucrose or glycerol biodegraded as a function of composting time determined from the non-dispersive infrared monitoring of carbon dioxide concentration in the dried gas stream exiting the bioreactor shown in FIG. 4A.

FIG. 5A is an exemplary plot of carbon dioxide concentration as a function of composting time determined using non-dispersive infrared monitoring of the dried gas stream exiting a bioreactor in which in which three 3M bamboo wipes are digested by bacteria obtained from a municipal yard waste inoculum.

FIG. 5B is an exemplary plot of mass percent of the three 3M bamboo wipes biodegraded as a function of composting time determined from the non-dispersive infrared monitoring of carbon dioxide concentration in the dried gas stream exiting the bioreactor shown in FIG. 5A.

While the above-identified drawings, which may not be drawn to scale, set forth various embodiments of the present disclosure, other embodiments are also contemplated, as noted in the Detailed Description. In all cases, this disclosure describes the presently disclosed invention by way of representation of exemplary embodiments and not by express limitations. It should be understood that numerous other modifications and embodiments can be devised by those skilled in the art, which fall within the scope and spirit of this invention.

### DETAILED DESCRIPTION

As used in this specification and the appended embodiments, the singular forms “a”, “an”, and “the” include plural referents unless the content clearly dictates otherwise. Thus, for example, reference to fine fibers containing “a compound” includes a mixture of two or more compounds. As used in this specification and the appended embodiments, the term “or” is generally employed in its sense including “and/or” unless the content clearly dictates otherwise.

As used in this specification, the recitation of numerical ranges by endpoints includes all numbers subsumed within that range (e.g. 1 to 5 includes 1, 1.5, 2, 2.75, 3, 3.8, 4, and 5). Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

Various exemplary embodiments of the disclosure will now be described with particular reference to the Drawings. Exemplary embodiments of the invention may take on various modifications and alterations without departing from the spirit and scope of the disclosure. Accordingly, it is to be understood that the embodiments of the invention are not to be limited to the following described exemplary embodiments, but is to be controlled by the limitations set forth in the claims and any equivalents thereof.

Thus, in one exemplary embodiments, the disclosure describes an apparatus, comprising:

a flow metering device for metering a flow of oxygen-containing gas to a humidifier,

a bioreactor comprising:

a body having an internal volume, a gas inlet connected with said bioreactor in flow communication between the humidifier and the internal volume to receive the flow of oxygen-containing gas in a humidified state from the humidifier, a dispersive element for distributing the flow of oxygen-containing gas in a humidified state throughout the internal volume, and a gas outlet in flow communication with the internal volume;

a first moisture trap in flow communication with the gas outlet;

a mass flow measuring device in flow communication with the first moisture trap; and

a non-dispersive infrared detector in flow communication with the mass flow measuring device, the non-dispersive infrared detector adapted to measure the concentration of carbon dioxide in a gas flowing from the first moisture trap.

In one exemplary embodiment, the apparatus further comprises:

at least three additional bioreactors, each additional bioreactor comprising:

a body having an internal volume, a gas inlet connected with said each additional bioreactor in flow communication between the humidifier and the internal volume to receive the flow of oxygen-containing gas in a humidified state from the humidifier,

a dispersive element for distributing the flow of oxygen-containing gas in a humidified state throughout the internal volume of the each additional bioreactor, and

a gas outlet in flow communication with the internal volume of the each additional bioreactor;

at least three additional first moisture traps, each additional first moisture trap in flow communication with the gas outlet of one of the each additional bioreactors; and

a multi-valve manifold in flow communication with each of the first moisture traps, the multi-valve manifold adapted to alternately direct gas from one of the first moisture traps in turn to the mass flow measuring device. In further exemplary embodiments of the foregoing embodiment, the apparatus further comprises a second moisture trap disposed between the multi-valve manifold and the non-dispersive infrared detector.

In additional exemplary embodiments of any of the foregoing aspect and embodiments, at least one of the dispersive elements is a mat of polymeric fibers. In certain such exemplary embodiments, the at least one of the dispersive elements is a fiber mesh mat of an interlacing network of polypropylene and polyester fibers. In further such exemplary embodiments, at least one of the bioreactors further comprises an element providing physical reinforcement to the at least one of the dispersive elements.

In further exemplary embodiments of any of the foregoing aspect and embodiments, the mass flow measuring device is an electronic flow meter disposed so as to measure the mass flow rate of the gas flowing from the first moisture trap to the non-dispersive infrared detector. In a presently preferred such embodiment, the apparatus further comprises a computer receiving information from the mass flow measuring device and the non-dispersive infrared detector, wherein the computer is used to calculate and report information on the reactions occurring in each bioreactor. In one such presently preferred embodiment, the apparatus further comprises a controller for operating the multi-valve manifold in response to timing signals from the computer.

In some presently preferred embodiments, the apparatus will have at least three additional bioreactors and at least three additional first moisture traps connected to these bioreactors. Such an arrangement allows a sample, a blank, a positive control, and a negative control to be tested simultaneously. An electronically-controlled multi-valve manifold for alternately directing gas from the bioreactors in turn to the mass-flow detector and the non-dispersive IR detector may be present in these embodiments. In any of the preceding aspect and embodiments, the internal volume of each bioreactor is from about 2 to 4 liters, and further the flow metering device delivers from about 100 to 150 ml of air per minute at ambient temperature and pressure to each bioreactor.

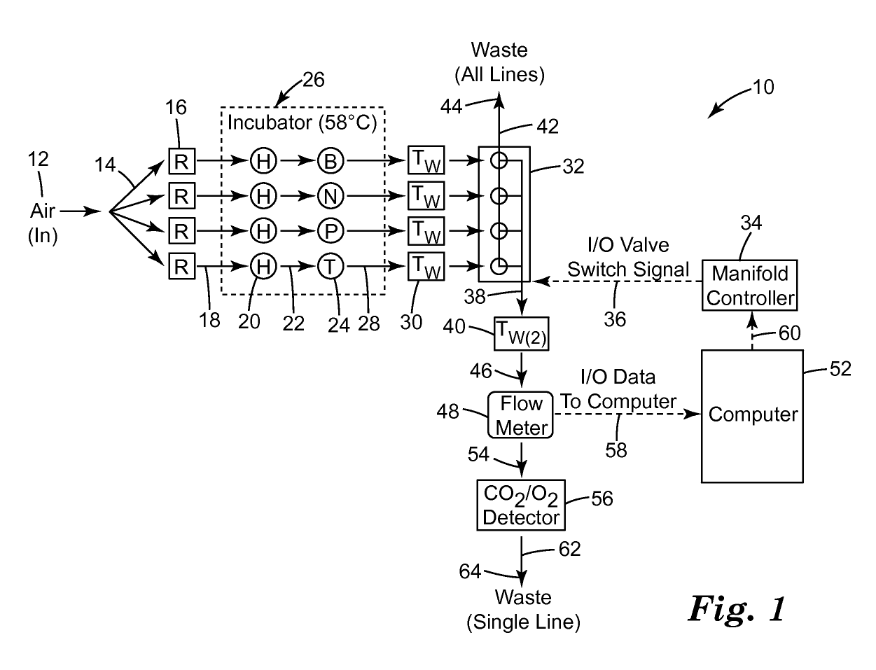

Referring now to FIG. 1, a schematic of an exemplary apparatus 10 according to the present disclosure is illustrated. The illustrated variant includes four copies of some of the components so that a sample, a blank, a positive control, and a negative control can be performed simultaneously. In convenient embodiments of the invention, additional components are present in groups of four so that additional samples can be tested and controlled for simultaneously.

The apparatus 10 is connected to a source of oxygen containing gas 12, conveniently air. Gas from the source 12 is routed through tube 14 to at least one mass flow controller or metering flowmeter 16, and from there through tube 18 to at least one humidifier 20. Humidified gas is conducted through tube 22 to bioreactor 24. In many convenient embodiments multiple copies of elements 16, 18, 20, 22, and 24 will be enclosed in a single incubator enclosure 26 to ensure that the bioreactors 24 will be operating in similar conditions.

Gas flowing out of the bioreactors 24, which may have had their compositions altered by biological processes within the bioreactors 24, are conducted by tube 28 to at least one moisture trap 30. In many convenient embodiments gas passing from the moisture trap 30 is directed through a computer-controllable multi-valve manifold 32 under the control of a device such as a programmable logic controller 34 via signal line 36.

Under the direction of the controller 34, gas from one bioreactor 24 at a time is conducted via tube 38 to an optional secondary moisture trap 40. Gas exiting all the non-selected bioreactors 24 is conducted via tube 42 to a waste vent 44.

Gas from the selected bioreactor 24 is conducted from the secondary moisture trap 40 via tube 46 to a sensing mass flow meter 48, conveniently one that can report electronically via sense line 50 to computer 52. After having been measured, the gas is conducted via tube 54 to a detector 56. The detector 56 includes a gas-phase infrared detector, conveniently a non-dispersive IR detector (NDIR) set at specific wavelengths for the measurement of the CO₂ in the gas, and conveniently a chemical cell detector for the measurement of the O₂ in the gas. Conveniently the detector 56 is one that can report electronically via sense line 58 to computer 52. Conveniently, the computer 52 can also time the actions of the controller 34 via control line 60. After being measured, the gas is conducted via tube 62 to waste vent 64.

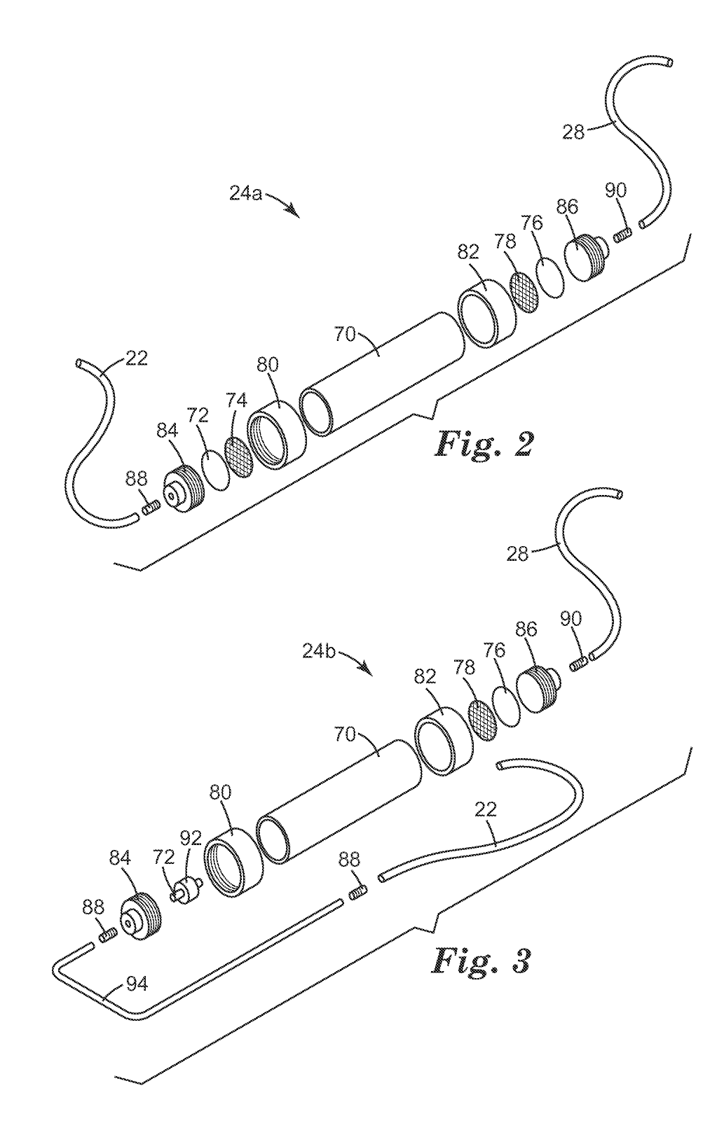

Referring now to FIG. 2, an exploded perspective view of a bioreactor 24a particularly adapted to the assessment of solid samples is illustrated. Bioreactor 24a includes a body 70 and a dispersive element 72 for distributing humidified gas flowing in through tube 22. Conveniently, the body 70 will be transparent so that the ongoing reaction can be observed. A body 70 constructed of transparent polyvinyl chloride pipe or transparent polyacrylate (e.g. Plexiglas®) 12 inches (30.5 cm) long and 4 inches (10.2 cm) in inside diameter has been found to be suitable. This provides a volume of approximately 3 liters within the bioreactor 24a. With a bioreactor 24a of such a size, good results are achieved when the metering flow meter 16 delivers between about 100 to 150 ml of air per minute to the bioreactor 24a. If the flow rate is too low, the compost is at risk of going anerobic. If the flow is too great, the environment within the bioreactor 24a may become too dry for the biodegradation reactions to proceed, and CO₂ levels may become too dilute to be accurately measured.

A polyolefin fiber mat has been found to be suitable for the dispersive element in the illustrated bioreactor 24a. More specifically, fiber mesh mat of an interlacing network of polypropylene and polyester fibers, commercially available as THINSULATE from 3M Company of St. Paul, Minn., has been used successfully. Physical reinforcement of the dispersive element 72, by an element such as e.g., a mesh of stainless steel wires 74 may be optionally employed. Similar elements, e.g., dispersive element 76 and mesh 78 may optionally be employed at the outlet of bioreactor 24a to further disperse the flow of oxygen-containing gas within the bioreactor 24a.

The bioreactor 24a is conveniently closed at both ends by threaded endpieces 80 and 82, conveniently solvent welded or adhesively bonded in place to body 70. Threaded caps 84 and 86 can then be used to openably close the ends of bioreactor 24a. The threaded caps 84 and 86 can be provided with tapped holes to receive threaded connectors 88 and 90 having barbed ends to connect to tubes 22 and 28.

Referring now to FIG. 3, an exploded perspective view of an alternate bioreactor 24b particularly adapted to the assessment of liquid samples is illustrated. Bioreactor 24b is conveniently constructed similarly to bioreactor 24a in FIG. 2, however in conveniently includes a stone sparger 92 as a substitute or as an adjunct to mesh 74. Suitable spargers 92 include Model 4450K (1-4) commercially available from McMaster-Carr of Santa Fe Springs, Calif. Further, good results are achieved by directing tube 22 into an elbow fitting 94, conveniently prepared from stainless steel tubing, such that inlet of the elbow fitting 94 is above the level of the fluid in the bioreactor 24b.

In another exemplary embodiment, the disclosure describes a method comprising:

(a) providing an apparatus comprising:
a flow metering device for metering a flow of oxygen-containing gas to a humidifier;
a bioreactor comprising a body having an internal volume, a gas inlet connected with said bioreactor in flow communication between the humidifier and the internal volume to receive the flow of oxygen-containing gas in a humidified state from the humidifier, a dispersive element for distributing the flow of oxygen-containing gas in a humidified state throughout the internal volume, and a gas outlet in flow communication with the internal volume;
a first moisture trap in flow communication with the gas outlet;
a mass flow measuring device in flow communication with the first moisture trap; and
a non-dispersive infrared detector in flow communication with the mass flow measuring device, the non-dispersive infrared detector adapted to measure the concentration of carbon dioxide in a gas flowing from the first moisture trap;

(b) adding to the internal volume of the bioreactor an at least partially compostable material, a liquid, and an inoculum comprising at least one bacterium;

(c) starting the flow of oxygen-containing gas to the bioreactor while maintaining the temperature of the bioreactor at a temperature sufficient to support asexual reproduction of the at least one bacterium, thereby producing a plurality of bacteria; and

(d) measure the concentration of carbon dioxide in the gas flowing from the first moisture trap over a time period sufficient to determine the rate at which the at least partially compostable material is digested by the plurality of bacteria.

In some exemplary embodiments of the foregoing method, the oxygen containing gas is air. In other exemplary embodiments, the at least partially compostable material comprises at least one of cellulose or a synthetic polymer. In additional exemplary embodiments, the liquid comprises water. In some particular exemplary embodiments, the inoculum comprises compost inoculums as specified in Section 9 of ASTM Test Method D 5338-98 (2003). In some particular exemplary embodiments, the temperature is selected to be from 25° C. to 65° C. In certain of these exemplary embodiments, the time period is from 24 hours to 2400 hours.

In some presently preferred embodiments, the internal volume of each bioreactor is from about 2 to 4 liters, and further wherein the flow metering device delivers from about 100 to 150 ml of air per minute at ambient temperature and pressure to each bioreactor. In certain of these embodiments, the mass flow measuring device is an electronic flow meter disposed so as to measure the mass flow rate of the gas flowing from the first moisture trap to the non-dispersive infrared detector. In additional such embodiments, the method further comprises a computer receiving information from the mass flow measuring device and the non-dispersive infrared detector, wherein the computer uses the information to calculate and reports the rate at which the at least partially compostable material is digested by the plurality of bacteria.

### EXAMPLES

An apparatus generally as depicted in FIG. 1 and as described above was constructed, except that 12 bioreactors and their associated humidifiers and flowmeters were used. For Example 1 involving the biodegradability testing of sucrose or glycerol, the bioreactor configuration of FIG. 3 was used. For Examples 2 and 3 involving the biodegradability testing of three 3M bamboo-cellulose kitchen wipes (available from 3M Co., St. Paul, Minn.), the bioreactor configuration of FIG. 2 was used.

In each Example, the source of oxygen-containing gas was compressed air, which was routed into metering flowmeters commercially available as 100-1000 cc/min VISI-FLOAT flowmeter from Dwyer Instruments, Inc. of Michigan City, Ind. Humidifiers of bubbler type were prepared from 250 ml HDPE bottles with screw caps. Bioreactors generally constructed as depicted in FIG. 2 were prepared with polyolefin fiber mat commercially available as THINSULATE from 3M being used as the dispersive element.

The humidifiers and bioreactors were mounted within an enclosure in the form of a cabinet with a front opening door. During operation, this enclosure was kept at a temperature of 58° C. First moisture traps were present, each having a capacity of 500 ml, and each being kept at a temperature of between 22 and 26° C. during the operation of the apparatus. The multi-valve manifold with temperature controller was constructed to order and given part number 104070 by Midac Corp. of Costa Mesa, Calif. This multi-valve manifold was kept at between about 60 and 80° C. during the operation of the apparatus in order to further reduce condensation of moisture within the manifold.

A secondary moisture trap was present downstream of the multi-valve manifold, and had a capacity of 250 ml and was operated at a temperature of between 22 and 26° C. during the operation of the apparatus. The sensing mass flow meter was a Model FMA-5606, commercially available from Omega Engineering, Inc. of Stamford, Conn., and it was cabled to send its information to a Model Aspire X-Series desktop microcomputer commercially available from Acer Computers of the Hypernet Group, LLC of Ingomar, Pa. Also sending its data to the microcomputer was the NDIR detector, a Model 9500 oxygen/carbon dioxide monitor, commercially available from Alpha-Omega Instruments, Corp. of Cumberland, R.I.

The microcomputer was running a data acquisition and control program written using LABVIEW visual programming software, commercially available as LABVIEW version 8 from National Instruments Corp. of Austin, Tex. The program operated to assess and report the CO₂ content and mass flow rate of the gas exiting each bioreactor as a function of time, associating a particular measurement at a particular time with a particular bioreactor internal volume from which the gas had exited. Additionally, the acquired data were recorded into a Microsoft EXCEL® template so as to provide convenient data manipulation.

The microcomputer also controlled an integrated programmable logic controller system comprised of part/model numbers FP-1000, FP-AI-110 and FP-DO-403, (commercially available from National Instruments Corp. of Austin, Tex.), so as to cycle the multi-valve manifold through its gas inputs. The microcomputer was set to allow 10 minutes for a sample to be taken, and cycled through the gas from each bioreactor continuously during operation of the apparatus. Power for the multi-valve manifold and some other components was provided by 24 volt and 12 volt commercially available as model numbers S82K-00724 and S82K-03012 from Omron, Inc of Kyoto, Japan.

For Examples 1-2 involving the compostability or biodegradability (e.g. sewerability) testing of sucrose or glycerol, respectively, the source of the inoculum containing at least one bacterium was activated sludge obtained as aqueous mixed liquor suspended solids (MLSS) obtained from the secondary aeration units at the Metro Wastewater Treatment Plant (St. Paul, Minn.; traditionally referred to as the Pig's Eye Plant). MSM obtained from an EPA biodegradation testing guidance OPPTS 835 has also been used. The suspended solids were allowed to settle under refrigerated conditions, and then the settled solids were transferred to a volume of mineral salts medium (MSM; about 25-100 mL of solids to ˜2 liter per 3 liter bioreactor internal volume). A presently preferred range is about 0.1 to 0.5 gram (dry weight) of solids per liter.

For Example 3 involving the compostability or biodegradability (e.g., landfillability) testing of three 3M bamboo kitchen wipes, the inoculum was selected to be composted municipal yard waste comprising cellulosic material (e.g. grass clippings, leaves, and other plant matter). The composted yard waste inoculum was obtained from municipal yard-waste compost that was collected from about May to November of 2009.

Other sources of inoculums can be used for the landfillability testing of Example 3, for example, commercial/industrial waste compost such as that derived from food-wastes, obtainable from a restaurant or landfill. Sources of that compost are more limited, but are available in some areas. However, municipal yard-waste compost is generally more readily available.

The composted yard waste inoculum was collected from windrows at the Woodbury, Minn., yard waste composting facility, from windrows that are evaluated for temperature with a 1 meter temperature probe. Compost from intermediate-stage degraded windrows with a temperature from ˜115° F. to 160° F. was used, with hotter compost typically being more active and desirable for use.

The composted yard waste inoculum was removed from windrows by hand using spade shovels and placed into 5 gallon buckets and sealed for return to the laboratory test apparatus. Typically 10-20 buckets were collected per trip. At the laboratory, the material was sieved to ≦1 cm particle size for use in the composting apparatus using an ASTM standard soil sieve. The moisture content of the compost was determined, and if the moisture content was less than 50% (w/w), water was mixed with the compost to bring the moisture content to about 50% (w/w) of the compost mixture (including the added water).

In Example 3, the composted yard waste inoculums was added to the internal volume of each of a plurality of bioreactors in the composting apparatus, by placing 1.00 kg of inoculum into each compost vessel (bioreactor) and mixing with the test substance (three 3M bamboo kitchen wipes), although it will be understood that other waste materials could be used for compostability testing, for example cellulose-containing packaging, synthetic plastic containers or other wastes to be evaluated for compostability or biodegradability.

One drop of inert anti-foam A (available from Dow Chemical Co., Midland, Mich.) was added to minimize foaming in the bioreactor vessel. A sparging stone in the bottom of each internal volume of each bioreactor aerates the internal volume with small air bubbles and its rate controlled by a flow measuring device and control valve.

The system was sealed and CO₂ liberated as a function of time as a result of the biodegradation of the test substance was measured by passing the dried bioreactor exhaust gas through a CO₂ measuring device, in this case, a non-dispersive infrared carbon dioxide gas analyzer (available from Alpha Omega Instruments, Inc., Cumberland, R.I.).

In each of Example 1-2, blank test runs (e,g, municipal yard waste inoculums or MSM Sludge without any added organic substance or test sample to be evaluated for compostability or biodegradability) were used to establish a zero baseline for CO₂ measurements. Positive controls (e.g. Examples 1-2), in which sucrose and glycerol were added to the internal volume of the bioreactor instead of the test samples may be used for comparative purposes. Exemplary results are shown in the Figures, in which:

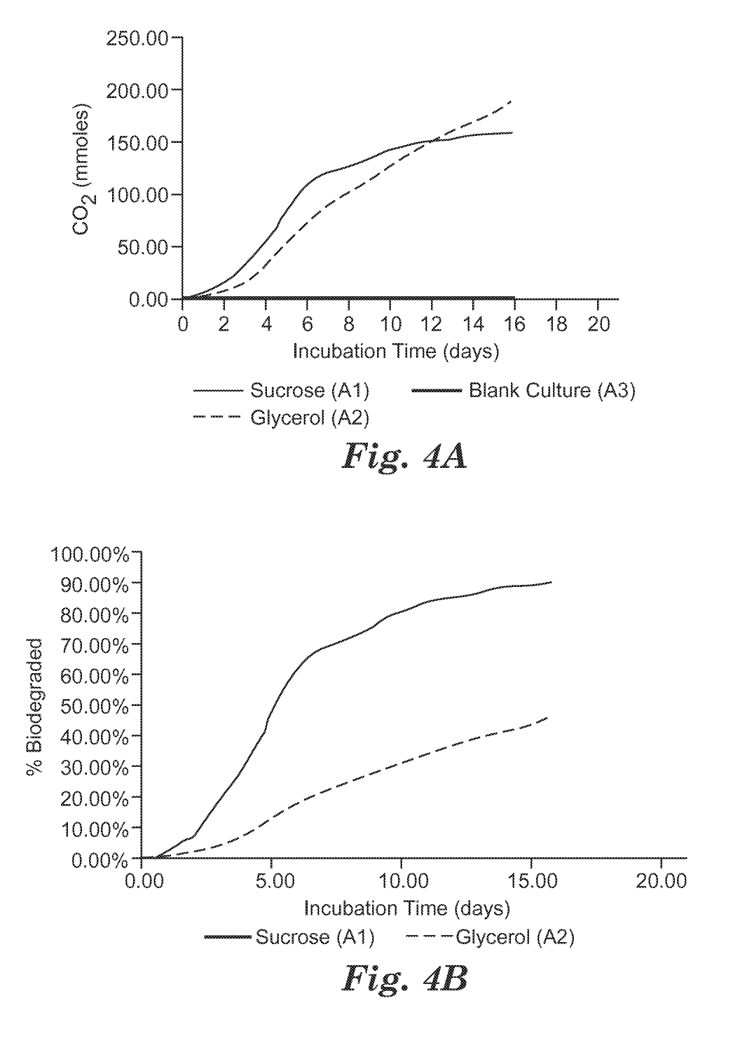

FIG. 4A is an exemplary plot of carbon dioxide concentration as a function of composting time determined using non-dispersive infrared monitoring of the dried gas stream exiting a bioreactor in which sucrose or glycerol are digested by bacteria obtained from a municipal sewage waste inoculum, according to Examples 1-2.

FIG. 4B is an exemplary plot of mass percent of the sucrose or glycerol biodegraded as a function of composting time determined from the non-dispersive infrared monitoring of carbon dioxide concentration in the dried gas stream exiting the bioreactor shown in FIG. 4A.

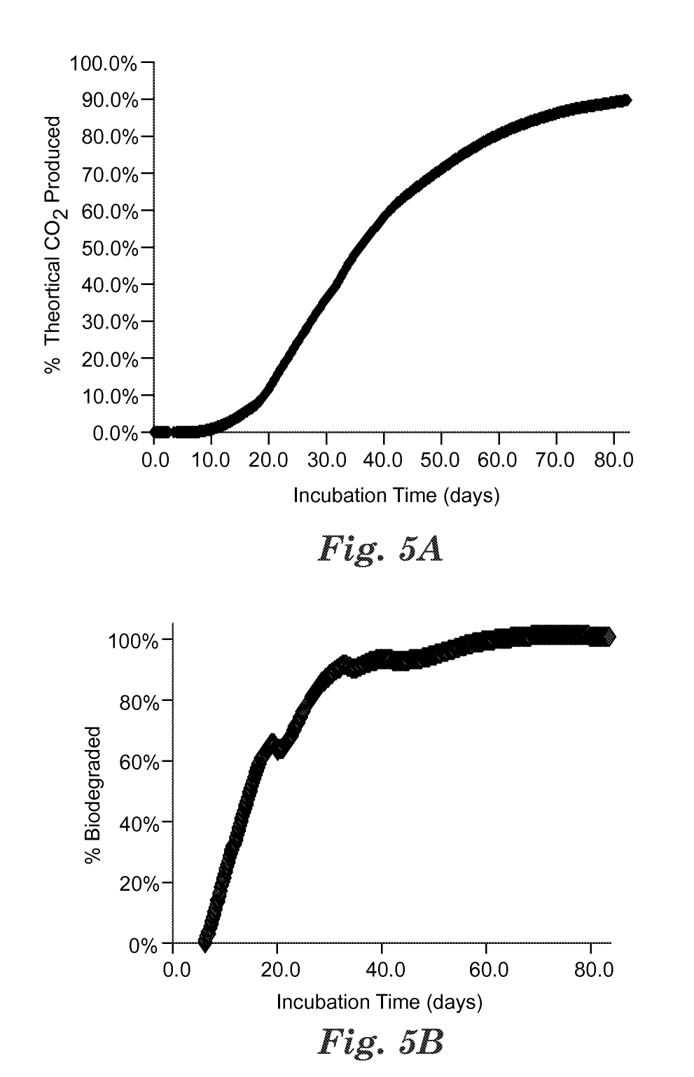

FIG. 5A is an exemplary plot of carbon dioxide concentration as a function of composting time determined using non-dispersive infrared monitoring of the dried gas stream exiting a bioreactor in which in which three 3M bamboo wipes are digested by bacteria obtained from a municipal yard waste inoculums, according to Example 3.

FIG. 5B is an exemplary plot of mass percent of the three 3M bamboo wipes biodegraded as a function of composting time determined from the non-dispersive infrared monitoring of carbon dioxide concentration in the dried gas stream exiting the bioreactor shown in FIG. 5A.

While the specification has described in detail certain exemplary embodiments, it will be appreciated that those skilled in the art, upon attaining an understanding of the foregoing, may readily conceive of alterations to, variations of, and equivalents to these embodiments. Accordingly, it should be understood that this disclosure is not to be unduly limited to the illustrative embodiments set forth hereinabove.

Furthermore, all publications, published patent applications and issued patents referenced herein are incorporated by reference in their entirety to the same extent as if each individual publication or patent was specifically and individually indicated to be incorporated by reference. Various exemplary embodiments have been described. These and other embodiments are within the scope of the following listing of disclosed embodiments.

What is claimed is:

1. An apparatus, comprising:
a flow metering device for metering a flow of oxygen-containing gas to a humidifier;
a bioreactor comprising a body having an internal volume, a gas inlet connected with said bioreactor in flow communication between the humidifier and the internal volume to receive the flow of oxygen-containing gas in a humidified state from the humidifier, a dispersive element for distributing the flow of oxygen-containing gas in a humidified state throughout the internal volume, and a gas outlet in flow communication with the internal volume;
a first moisture trap in flow communication with the gas outlet;
a mass flow measuring device in flow communication with the first moisture trap; and
a non-dispersive infrared detector in flow communication with the mass flow measuring device, the non-dispersive infrared detector adapted to measure the concentration of carbon dioxide in a gas flowing from the first moisture trap.

2. An apparatus according to claim 1, further comprising:
at least three additional bioreactors, each additional bioreactor comprising:
a body having an internal volume, a gas inlet connected with said each additional bioreactor in flow communication between the humidifier and the internal volume to receive the flow of oxygen-containing gas in a humidified state from the humidifier,
a dispersive element for distributing the flow of oxygen-containing gas in a humidified state throughout the internal volume of the each additional bioreactor, and
a gas outlet in flow communication with the internal volume of the each additional bioreactor;
at least three additional first moisture traps, each additional first moisture trap in flow communication with the gas outlet of one of the each additional bioreactors; and
a multi-valve manifold in flow communication with each of the first moisture traps, the multi-valve manifold adapted to alternately direct gas from one of the first moisture traps in turn to the mass flow measuring device.

3. An apparatus according to claim 2, further comprising a second moisture trap disposed between the multi-valve manifold and the non-dispersive infrared detector.

4. An apparatus according to claim 1, wherein at least one of the dispersive elements is a mat of polymeric fibers.

5. An apparatus according to claim 4, wherein the at least one of the dispersive elements is a fiber mesh mat of an interlacing network of polypropylene and polyester fibers.

6. An apparatus according to claim 5, wherein at least one of the bioreactors further comprises an element providing physical reinforcement to the at least one of the dispersive elements.

7. An apparatus according to claim 1, wherein the mass flow measuring device is an electronic flow meter disposed so as to measure the mass flow rate of the gas flowing from the first moisture trap to the non-dispersive infrared detector.

8. An apparatus according to claim 7, further comprising a computer receiving information from the mass flow measuring device and the non-dispersive infrared detector, wherein the computer is used to calculate and report information on the reactions occurring in each bioreactor.

9. An apparatus according to claim 8, further comprising a controller for operating the multi-valve manifold in response to timing signals from the computer.

10. An apparatus according to claim 9, wherein the internal volume of each bioreactor is from about 2 to 4 liters, and further wherein the flow metering device delivers from about 100 to 150 ml of air per minute at ambient temperature and pressure to each bioreactor.

11. A method comprising:
(a) providing an apparatus comprising:
a flow metering device for metering a flow of oxygen-containing gas to a humidifier;
a bioreactor comprising a body having an internal volume, a gas inlet connected with said bioreactor in flow communication between the humidifier and the internal volume to receive the flow of oxygen-containing gas in a humidified state from the humidifier, a dispersive element for distributing the flow of oxygen-containing gas in a humidified state throughout the internal volume, and a gas outlet in flow communication with the internal volume;
a first moisture trap in flow communication with the gas outlet;
a mass flow measuring device in flow communication with the first moisture trap; and
a non-dispersive infrared detector in flow communication with the mass flow measuring device, the non-dispersive infrared detector adapted to measure the concentration of carbon dioxide in a gas flowing from the first moisture trap;
(b) adding to the internal volume of the bioreactor an at least partially compostable material, a liquid, and an inoculum comprising at least one bacterium;
(c) starting the flow of oxygen-containing gas to the bioreactor while maintaining the temperature of the bioreactor at a temperature sufficient to support asexual reproduction of the at least one bacterium, thereby producing a plurality of bacteria; and
(d) measure the concentration of carbon dioxide in the gas flowing from the first moisture trap over a time period sufficient to determine the rate at which the at least partially compostable material is digested by the plurality of bacteria.

12. The method according to claim 11, wherein the oxygen containing gas is air.

13. The method according to claim 11, wherein the at least partially compostable material comprises at least one of cellulose or a synthetic polymer.

14. The method according to claim 11, wherein the liquid comprises water.

15. The method according to claim 11, wherein the inoculum comprises compost inoculums as specified in Section 9 of ASTM Test Method D 5338-98 (2003).

16. The method according to claim 11, wherein the temperature is selected to be from 25° C. to 65° C.

17. The method according to claim 11, wherein the time period is from 24 hours to 2400 hours.

18. The method according to claim 11, wherein the internal volume of each bioreactor is from about 2 to 4 liters, and further wherein the flow metering device delivers from about 100 to 150 ml of air per minute at ambient temperature and pressure to each bioreactor.

19. The method according to claim 11, wherein the mass flow measuring device is an electronic flow meter disposed so as to measure the mass flow rate of the gas flowing from the first moisture trap to the non-dispersive infrared detector.

20. The method according to claim 19, further comprising a computer receiving information from the mass flow measuring device and the non-dispersive infrared detector, wherein the computer uses the information to calculate and reports the rate at which the at least partially compostable material is digested by the plurality of bacteria.
